(19) United States
(12) Patent Application Publication
(10) Pub. No.: US 2021/0164046 A1
(43) Pub. Date: Jun. 3, 2021

(54) Nucleic Acid Coated Micron and Submicron Particles for Authentication

(71) Applicant: APDN (B.V.I.) Inc., Tortola (VG)

(72) Inventors: Michael E. HOGAN, Stony Brook, NY (US); Lawrence JUNG, Dix Hills, NY (US); Yuhua SUN, Stony Brook, NY (US); Maciej Szczepanik, Mount Sinai, NY (US)

(21) Appl. No.: 17/175,938

(22) Filed: Feb. 15, 2021

Related U.S. Application Data
(63) Continuation-in-part of application No. 15/890,541, filed on Feb. 7, 2018, now Pat. No. 10,920,274.
(60) Provisional application No. 62/461,312, filed on Feb. 21, 2017.

(51) Int. Cl. C12Q 1/6876; C12Q 1/6844

(52) U.S. Cl. CPC C12Q 1/6876; C12Q 2563/185; C12Q 1/6844

(57) ABSTRACT

A composition comprising micron or submicron particles covered by a monolayer of nucleic acid wherein the nucleic acid may be recovered from the submicron particles is claimed. Methods of attaching a nucleic acid to an object for authentication and methods of authenticating an object are also claimed.

### CROSS-REFERENCE TO RELATED APPLICATION

This application claims the benefit of U.S. Provisional Application No. 62/461,312, filed on Feb. 21, 2017, which is hereby incorporated by reference in its entirety. This application is also a continuation in part of U.S. patent application Ser, No. 15/890,541 filed on Feb. 7, 2018, which is also hereby incorporated by reference in its entirety.

### BACKGROUND OF THE INVENTION

Nucleic acids have been permanently conjugated to metal oxides and other particles for use as biosensors and for biomedical diagnosis, therapy, and catalysis. These metal oxides and other particles are often powders comprised of micron or sub-micron diameters sized particles. Additionally, DNA has been used as a taggant for purposes of authenticating objects. For example, in U.S. Pat. No. 9,297,032, DNA is mixed with a perturbant and a polymer to coat an object. The DNA may be recovered from the object and PCR-based assays are performed to verify the taggant, thus authenticating the object.

However, there remains a need to incorporate nucleic acid taggants into materials that cannot be introduced into water, i.e., water immiscible materials which are not or cannot be produced using water, or materials in which the raw materials are often comprised of powders, e.g., pharmaceuticals, cosmetics and nutraceuticals. In addition, there remains a further unmet need for a method of removably attaching a nucleic acid taggant to a micron or submicron particle, such that the nucleic acid can be later readily removed from the particle for the purpose of authentication. A preferred application for such removable attachment is for powders used in the pharmaceutical, cosmetic and nutraceutical industries.

### SUMMARY OF THE INVENTION

The present inventors have found a means of removably affixing a layer or monolayer of nucleic acid onto the surface of micron or submicron particles so that the nucleic acid may be later readily recovered and isolated from the micron or submicron particle. These DNA tagged micron or submicron particles can then be used to form powders which are commonly used in the pharmaceutical, cosmetic and nutraceuticals industries.

In one embodiment, the invention relates to a composition including micron or submicron particles covered by a monolayer of nucleic acid, wherein the nucleic acid may be recovered from the submicron particles. The nucleic acid is preferably affixed to the submicron particles. In an alternative embodiment the invention relates to a composition including micron or submicron particles covered in a layer of nucleic acid, wherein the nucleic acid may be recovered from the micron or submicron particles. The nucleic acid layer is preferably affixed to the micron or submicron particles.

The preferred nucleic acid is deoxyribonucleic acid (DNA). The preferred micron or submicron particles are, without limitation, metal oxides, dicalcium phosphate, rice flower, rice husk, silicone dioxide, maltodextrin, magnesium, vegetable stearate, ethyl cellulose, silica, diatomaceous earth, sodium benzoate, antioxidants, pectin, sodium citrate and citrate. Preferred metal oxides are titanium dioxide and silicon dioxide.

The micron or submicron particles may be exposed to a substance to optimize the desired level of adhesion of nucleic acid to submicron particles so the nucleic acid may be recovered from the submicron particles. Preferably, the substance is selected from the group consisting of sodium phosphate, borate, monopotassium phosphate, vanadate, citrate, ethylenediaminetetraacetic acid, sodium dodecyl sulfate, and sodium lauryl sulfate.

In an embodiment, the invention relates to a method of attaching a nucleic acid to an object for authentication purposes comprising providing a plurality of micron or submicron particles; adding an amount of nucleic acid suspended in a solvent to the submicron particles so that only enough nucleic acid is present to form a monolayer around each submicron particle; extracting the solvent to form a monolayer of nucleic acid covering each submicron particle; and attaching the nucleic acid covered submicron particles to an object to be authenticated using nucleic acid amplification and/or taggant sequence detection techniques for authentication. Preferably the solvent is water.

In another embodiment, the invention relates to a method of attaching a nucleic acid to an object for authentication purposes comprising providing a plurality of micron or submicron particles; adding an amount of nucleic acid suspended in a solvent so that only enough nucleic acid is present to form a monolayer around each submicron particle; spray drying the plurality of micron or submicron particles and the nucleic acid suspended in a solvent to form a plurality of micron or submicron particles with a monolayer of nucleic acid covering each submicron particle; and attaching the nucleic acid covered micron or submicron particles to an object to be authenticated using nucleic acid amplification and/or taggant sequence detection techniques for authentication. Preferably the solvent is water or any other suitable solvent.

In another embodiment, the invention relates to a method of attaching a nucleic acid to an object for authentication purposes comprising providing a plurality of micron or submicron particles; adding an amount of nucleic acid suspended in a solvent; spray drying the plurality of micron or submicron particles and the nucleic acid suspended in a solvent to form a plurality of micron or submicron particles coated with a layer of nucleic acid; and attaching the nucleic acid covered micron or submicron particles to an object to be authenticated using nucleic acid amplification and/or taggant sequence detection techniques for authentication. Preferably the solvent is water or any other suitable solvent.

In another embodiment, the invention relates to a method of authenticating an object comprising providing a plurality of micron or submicron particles; adding an amount of nucleic acid suspended in a solvent so that only enough nucleic acid is present to form a monolayer around each submicron particle; spray drying the plurality of micron or submicron particles and the nucleic acid suspended in a solvent to form a plurality of micron or submicron particles with a monolayer of nucleic acid covering each submicron particle; attaching the nucleic acid covered micron or submicron particles to an object to be authenticated; taking a sample of the object to recover the nucleic acid from the micron or submicron particles; isolating the nucleic acid; amplifying and identifying the nucleic acid using nucleic acid amplification and/or taggant sequence detection techniques; and verifying the authenticity of the object by the presence of the nucleic acid.

In another embodiment, the invention relates to a method of authenticating a pharmaceutical or nutraceutical product comprising providing plurality of micron or submicron particles; adding an amount of nucleic acid suspended in a solvent so that only enough nucleic acid is present to form a monolayer around each submicron particle; spray drying the plurality of micron or submicron particles and the nucleic acid suspended in a solvent to form a plurality of micron or submicron particles with a monolayer of nucleic acid covering each submicron particle; adding the nucleic acid covered micron or submicron particles to a pharmaceutical or nutraceutical product; taking a sample of the pharmaceutical or nutraceutical product to recover the nucleic acid from the micron or submicron particles disposed on or within said product; isolating the nucleic acid; amplifying and identifying the nucleic acid using nucleic acid amplification and/or taggant sequence detection techniques; and verifying the authenticity of the pharmaceutical or nutraceutical product by the presence of the nucleic acid. The nucleic acid may be DNA. The micron or submicron particles are any suitable powdered excipient for a pharmaceutical or nutraceutical product or may be, without limitation, citrate, sodium citrate, titanium dioxide, maltodextrin, hydroxypropyl methylcellulose. The solvent may be water or any other suitable solvent.

### BRIEF DESCRIPTION OF THE FIGURES

The accompanying figures, which are incorporated into and form a part of the specification, illustrate one or more embodiments of the present invention and, together with the description, serve to explain the principles of the invention. The figures are only for the purpose of illustrating the preferred embodiments of the invention and are not to be construed as limiting the invention. In the figures:

FIG. 1 is a bar chart showing levels of DNA taggant detection by qPCR, a taggant specific detection technique, when DNA taggants are spray dried onto citrate/sodium citrate micron or submicron particles according to an embodiment of the invention.

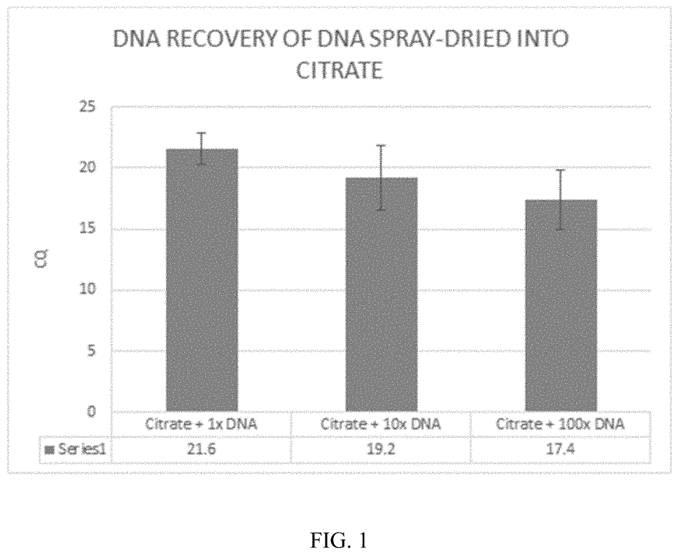

FIG. 2 is a bar chart showing levels of DNA taggant detection by qPCR, a taggant specific detection technique, when DNA taggants are spray dried onto citrate and maltodextrin micron or submicron particles according to an embodiment of the invention.

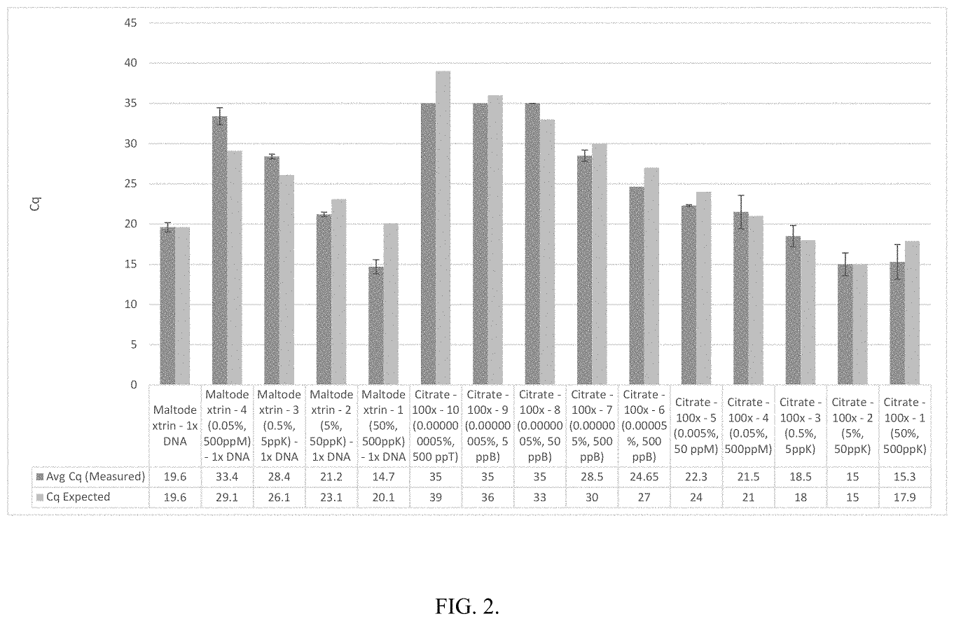

FIG. 3 is a bar chart showing levels of DNA taggant detection by qPCR, a taggant specific detection technique, when DNA taggants are spray dried onto titanium dioxide (Ti02) or hypromellose (hydroxypropyl methylcellulose) micron or submicron particles according to an embodiment of the invention.

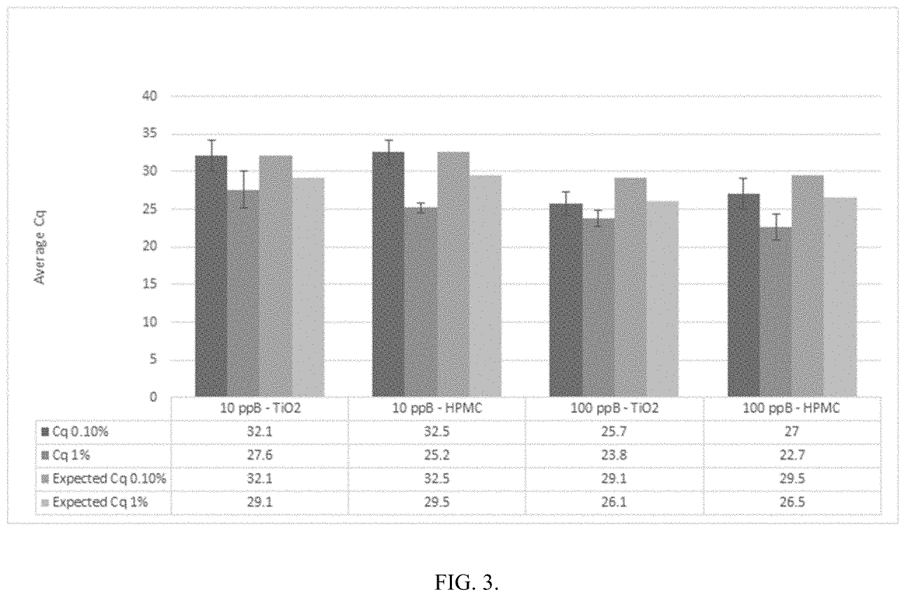

### DETAILED DESCRIPTION

A composition including micron or submicron particles covered by a removably affixed monolayer of nucleic acid taggants is claimed. The nucleic acid taggant may be readily or otherwise removed from the micron or submicron particles so the nucleic acid taggant may be amplified and identified using nucleic acid amplification and/or taggant sequence detection techniques.

Submicron particles measure under 1 μm (1,000 nm) in diameter. The submicron particles of the invention include any submicron particle that can be incorporated within an object or attached to an object. Preferred submicron particles are spherical, have known circumferences, disburse well in water, and provide advantageous binding conditions for a nucleic acid, and include powdered pharmaceutical and/or nutraceutical excipients.

Micron particles measure approximately 1 μm in diameter. As used in this application, a micron particle may be up to 3 μm in diameter. The micron particles of the invention include any micron particle that can be incorporated within an object or attached to an object. Preferred micron particles are spherical, have known circumferences, disburse well in water, and provide advantageous binding conditions for a nucleic acid, and include powdered pharmaceutical and/or nutraceutical excipients.

Examples of micron or submicron particles include metal oxides, metal carbides, metal nitrides, metal sulfates dicalcium phosphate, rice flower, rice husk, silicone dioxide, maltodextrin, magnesium, vegetable stearate, ethyl cellulose, silica, hydroxypropyl methylcellulose, diatomaceous earth, sodium benzoate, antioxidants, hydroxypropyl methylcellulose, pectin sodium citrate and citrate. Preferred metal oxides are titanium dioxide and silicon dioxide. The preferred micron or submicron particles are metal oxides, citrate, maltodextrin and pectin. The micron and submicron particles may be incorporated into pharmaceuticals, foods, cosmetics and nutraceuticals as excipients or active ingredients. In addition, the micron and/or submicron parties can be incorporated into most commercially available materials including, for example, thermoplastics, acrylics, textiles, and polymers, without causing adverse structural effects.

The nucleic acid is used as a taggant, i.e., a substance that is affixed to an object to provide information about the object such as the source of manufacture, national origin, or authenticity. “Nucleic acid” and “nucleic acid taggant” are used interchangeably throughout the application. Nucleic acid includes DNA and ribonucleic acid (RNA). Preferably, the nucleic acid taggant is a non-naturally occurring sequence that is adapted for use in authentication. The preferred nucleic acid is DNA.

Nucleic acid taggants useful in the invention include any suitable nucleic acid taggant, including DNA taggants. In one example, the DNA taggant is a double stranded DNA molecule having a length of between about 20 base pairs and about 1000 base pairs. In another example, the DNA taggant is a double-stranded DNA molecule with a length of between about 80 and 500 base pairs. In another example, the DNA taggant is a double-stranded DNA molecule having a length of between about 100 and about 250 base pairs. Alternatively, the DNA taggant can be single-stranded DNA of any suitable length, such as between about 20 bases and about 1000 bases; between about 80 bases and 500 bases; or between about 100 bases and about 250 bases. The DNA taggant can be a naturally-occurring DNA sequence, whether isolated from natural sources or synthetic; or the DNA taggant can be a non-naturally occurring sequence produced from natural or synthetic sources. All or a portion of the DNA may comprise an identifiable sequence. The preferred DNA is double-stranded DNA of a non-naturally occurring sequence. The DNA taggant may be comprised of an amplicon produced via the polymerase chain reaction (PCR). The DNA taggant may alternatively by comprised of a mixture of amplicons produced via the polymerase chain reaction (PCR) and oligonucleotides produced via sold-state oligonucleotide synthesis.

Preferably, the DNA taggant is identifiable by any suitable nucleic acid amplification and/or taggant sequence detection technique. Nucleic acid amplification may be accomplished via any technique known in the art, such as, for example, polymerase chain reaction (PCR), loop mediated isothermal amplification, rolling circle amplification, nucleic acid sequence base amplification, ligase chain reaction, or recombinase polymerase amplification. In addition, any known sequence detection and/or identification technique may be used to detect the presence of the nucleic acid taggant such as, for example, hybridization with a taggant-sequence specific nucleic acid probe, an in situ hybridization method (including fluorescence in situ hybridization: FISH), as well as amplification and detection via PCR, such as quantitative (qPCR)/real time PCR (RT-PCR). Isothermal amplification and taggant sequence detection may also be performed. Digital PCR, which results in extremely high sensitivity and accuracy and may use a nanofluidic chip may also be utilized as a taggant sequence detection technique.

In order to identify the nucleic acids (which comprise a DNA taggant), and thus authenticate an associated object, it is important that the nucleic acids be readily removable from the object to which they are applied. In other words, enough nucleic acid must be removable from the object to enable nucleic acid amplification and/or taggant sequence detection techniques. Removal of nucleic acids from an object may be performed via the removal of nucleic acids from the surface of the object without the removal of the nucleic acids' associated submicron particle. Removal of nucleic acids may also be accomplished via the removal of one or more nucleic acid-coated submicron particles attached to an object. The nucleic acid is then disassociated from the recovered submicron particle(s), as described herein, so that the nucleic acids can be amplified and identified using nucleic acid amplification and/or taggant sequence detection techniques. Alternatively, the micron or submicron particles coated with nucleic acid may be dissolved, along with the object desired to be authenticated and the result solution subject to nucleic acid amplification and/or taggant sequence detection techniques.

“Readily removing the nucleic acid from the object to which it was applied” is defined as removing the nucleic acid and/or DNA-coated submicron particles in a manner that is not laborious. For example, “readily removing the nucleic acid from the object to which it was applied” includes wiping the surface of the object the nucleic acid-covered submicron particles are attached to with a wet cotton swab. In another example, “readily removing the nucleic acid from the object to which it was applied” includes using a cotton swab with methyl ethyl ketone to wipe the object. In an additional example, “readily removing the nucleic acid from the object to which it was applied” includes using a competitive binding substance to detach the nucleic acids- or DNA-coated submicron particles from the object. In another example, “readily removing the nucleic acid from the object to which it was applied” includes dissolving the object containing the nucleic acid coated micron or submicron particles in a solution.

In order to allow the nucleic acid to be readily removed from the micron or submicron particles, the particles may be treated with a competitive binding substance to optimize the desired level of adhesion of nucleic acid to the micron or submicron particles before the nucleic acid is affixed to the particles. The micron or submicron particles may be treated with the competitive binding substance before or after the nucleic acid is affixed to the micron or submicron particles. Optimal adhesion would allow for the nucleic acid taggant to adhere to the micron or submicron particles so that the taggant remains affixed throughout the particles' lifecycle, but the adhesion cannot be so strong that not enough nucleic acid taggant can be removed from the micron or submicron particles to allow for the use of nucleic acid amplification and/or taggant sequence detection techniques when authentication is later desired.

For example, titanium dioxide is known to bond strongly to a nucleic acid. As a result, it is difficult to remove the nucleic acid affixed to an untreated titanium dioxide submicron particle when authentication is desired. In addition, due to the high level of adhesion between nucleic acid and untreated titanium dioxide, the nucleic acid can be damaged during the removal process. To address this problem, a titanium dioxide submicron particle may be treated with a competitive binding substance to reduce the submicron particle's bonding strength vis-a-vis nucleic acid such that when the titanium dioxide submicron particles are exposed to the nucleic acid taggant, the bonding forces between the nucleic acid and the titanium dioxide submicron particles will be permanently weakened, thus allowing for the ready removal of the nucleic acids when authentication is desired.

Nucleic acids bind to metal oxide submicron particles via the non-covalent bonding of the nucleic acid's phosphate backbone to the metal oxides' surface hydroxyl groups. An advantageous competitive binding substance to pre-treat the metal oxide submicron particles to aid in nucleic acid recovery for authentication may be any substance that will competitively bond to the surface hydroxyl groups of the metal oxide submicron particles, thus reducing overall non-covalent bonding strength between the nucleic acid and the metal oxide micron or submicron particle. Preferred competitive binding substances include sodium phosphate, borate, vanadate, citrate, ethylenediaminetetraacetic acid, monopotassium phosphate, sodium dodecyl sulfate, and sodium lauryl sulfate. The competitive binding substances may be used before or after a micron or submicron particle is introduced to nucleic acids. A micron or submicron particle may also be treated with a competitive binding substance after the formation of the nucleic acid monolayer to facilitate the removal of the nucleic acid from the submicron particle, and to also inhibit the nucleic acid from rebinding to a submicron particle at the time of authentication. A substance may also be used to pre-treat submicron particles that do not bond well to nucleic acids, or if the binding strength of nucleic acids needs to be increased. In one embodiment titanium dioxide submicron particles may be treated with hydrochloric acid or other acids to increase binding strength by protonating oxygen.

A method of covering micron or submicron particles with a monolayer of nucleic acid involves providing a plurality of uniform submicron particles. The micron or submicron particles may be treated with a competitive binding substance to optimize the desired level of adhesion of nucleic acid to submicron particles as discussed above. Alternatively, the micron or submicron particles may be treated with an acid such as hydrochloric acid to increase nucleic acid binding strength. Then, nucleic acid suspended in a solvent, preferably water at a pH <4, is added to the micron or submicron particles. The nucleic acid solution and submicron particles may be combined by methods known in the art such as stirring, vortexing, agitating, or centrifuging. Adding the correct amount of nucleic acid molecules to the solution is important for creating a monolayer of nucleic acid around each submicron particle. The correct amount of nucleic acid molecules in the solution is the exact amount of nucleic acid molecules necessary to form a monolayer of nucleic acid around each micron or submicron particle, based upon the calculated surface area of the micron or submicron particles and the nucleic acid molecules. These surface areas may be calculated by the methods described below. The competitive binding substance may also be applied after the nucleic acid is introduced to the micron or submicron particles.

The total surface area of a known mass of spherical micron or submicron particles may be calculated by using the size, i.e., diameter of the particles. The surface area of a sphere is 41⁻Ir², where r is the radius of the submicron particle, i.e., half of the diameter. If the mass of an individual micron or submicron particle is known, the total number of micron or submicron particles in the total mass can then be calculated. Therefore, the total surface area of a mass of uniform spherical micron or submicron particles can be calculated.

Likewise, the surface area of a nucleic acid molecule can be calculated based upon the number of base pair in a specific sequence. In regards to B-DNA (the most common form of DNA), a base pair is 3.4 A in length. The approximate width of double stranded B-DNA is 20 A. The length and width of all other forms of nucleic acids are also known. Therefore, the number of nucleic acid molecules necessary to create a monolayer around each micron or submicron particle can be calculated by dividing the surface area of the particle by the surface area of the nucleic acid sequence. This number of nucleic acids can then be multiplied by the number of micron or submicron particles in a given mass. The calculated number of nucleic acid molecules can then be converted into a mass quantity via known methods of calculation or by directly measuring with known devices.

The precise number of nucleic acid molecules in a solution can be accurately measured using known methods and devices. Devices such as the Bioanalyzer (Agilent Technologies, United States), the Qubit (ThermoFisher Scientific, United States) and/or the Nanodrop (Thermo Scientific, United States) can precisely measure nucleic acid concentrations in a solution, and thus, the number of nucleic acid molecules in a solution. Any other suitable instrument for the quantification of DNA in a solution may also be used. In addition, qPCR can be used to determine the absolute quantification of the number of nucleic acid molecules in a solution through known methods. The duration and extent of combining the nucleic acid solution with the micron or submicron particles may be determined by a person having ordinary skill in the art so that the nucleic acid may form a monolayer about each particle.

After a monolayer of nucleic acid is formed about each micron or submicron particle, the solvent may be removed by known techniques such as vacuum, centrifuge, heating, evaporation, use of a desiccant, and the like. The resulting product is a monolayer of nucleic acid covering each micron or submicron particle.

Alternately, a layer or monolayer of nucleic acid (DNA taggant) can be formed around a micron or submicron particle via the use of a spray dryer. In this embodiment, the appropriate amount of nucleic acid in solution is deposed onto the micron or submicron particles via a spray drying process to create a powder of micron or submicron particles coated with a layer or monolayer of DNA taggants. For the spray drying process, a slurry (liquid feed) comprised of the micron or submicron particles and the proper quantity of DNA taggants in solution is formed. This slurry is then processed in a spray drying apparatus to form a powder of micron or submicron particles with a layer or monolayer of DNA taggants disposed on each particle's exterior surface. Suitable micron or submicron particles for the spray dry disposition of a layer of monolayer of DNA taggants include, without limitation, metal oxides, dicalcium phosphate, rice flower, rice husk, silicone dioxide, maltodextrin, magnesium, vegetable stearate, ethyl cellulose, silica, diatomaceous earth, sodium benzoate, antioxidants, pectin, sodium citrate and citrate. Preferred particles are titanium dioxide, citrate, maltodextrin, hydroxypropyl methylcellulose and silica. Alternatively, spray drying can also be used to create a powder comprised of micron or submicron particles wherein the particles have the DNA taggants disposed within the particles. In this embodiment, the disposition of the DNA taggants is throughout the particle, not just on the exterior surface.

The DNA taggants may also be added as a layer or monolayer to micron or submicron particles during the spray drying process wherein during the manufacturing process of a powder comprised of micron or submicron particles, the appropriate amount of DNA taggants in solution are added to the spray drying apparatus not as part of the slurry/liquid feed. In this process, the DNA taggants are added after the formation of the micron or submicron particles in the spray dry process, and thus ensures application of the DNA taggants to the exterior of the particle. An already formed powder may be placed in a spray dry apparatus for the specific purpose of applying of DNA taggants to its particles' external surface, which would be applied via solution in the spray dry apparatus. Alternately, after the spray dry process, the DNA taggants may be applied in a solution to the formed micron or submicron power or the DNA taggants may be dry blended. The DNA taggants may be added at any concentration to the micron or submicron particles. Exemplary concentrations include 1 part-per-million, 1 part-per-billion and 100 parts-per-billion. The following w/w concentrations of DNA taggant to micron or submicron particle are preferred: 1.0g DNA taggant per kg of micron or submicron particle; 0.001g DN A per kg of micron or submicron particle and 0.1g of DNA taggant per kg of micron or submicron particle.

The nucleic acid covered micron or submicron particles may then be attached to an object. The relative quantity of nucleic acid micron or submicron particles attached to an object may vary based upon the target object's material, manufacturing process, storage conditions, use conditions, exposure to ultra violate light, or other variables that may affect the integrity of nucleic acids. Any means of attaching the nucleic acid covered micron or submicron particles to an object may be employed, including any known method of attaching micron or submicron particles to an object. In a preferred embodiment, the nucleic acid covered submicron particles may be included in a pharmaceutical or nutraceutical composition as an excipient. The final pharmaceutical or nutraceutical composition (object) may be in any form, including without limitation a solid oral dosage form, a liquid or a powder. In another example, the nucleic acid covered micron or submicron particles may be included in a cosmetic composition as an active ingredient. The nucleic acid covered micron or submicron particles may also be included into the master batch of thermoplastic or acrylic based materials such that the final product contains the micron or submicron particles. Furthermore, the nucleic acid covered micron or submicron particles may be included into any water immiscible solutions and/or water prohibitive materials such as cyanoacrylates, polyurethane, lacquers, shellacs, epoxy based-compounds, and acrylic compounds. Alternatively, the nucleic acid covered micron or submicron particles may be attached to the outside of an object or incorporated into the material that comprises the object.

The object may then be authenticated at a later time. Authentication of the object may involve removing a quantity of nucleic acid from the micron or submicron particles attached to the object. As mentioned above, it is preferable that the nucleic acid is readily removed from the micron submicron particles and the object. Methods of removing the nucleic acid from the submicron particles are known. Some methods of removing the nucleic acid are discussed above. In one embodiment, the material of the object may be dissolved by a solvent in order to remove one or more micron or submicron particles from the object. The nucleic acid on the recovered submicron particles may then be removed from the particle(s) and isolated. In one embodiment, the nucleic acid is removed from the micron or submicron particles by using a solution containing a high concentration of a competitive binding substance. The high concentration of competitive binding substance causes the nucleic acids to release from the micron or submicron particles and inhibits the nucleic acids from rebinding to the particles, thus allowing the nucleic acids to stay in solution. In the case where the micron or submicron particles are soluble in a solution, a solution may be utilized to dissolve the particles and release the nucleic acid into solution. When the nucleic acid is removed from the micron or submicron particles in a solution, the solution is then utilized for identifying the nucleic acid via nucleic acid amplification and/or taggant sequence detection techniques.

Once the nucleic acid is removed from the micron or submicron particles and isolated, nucleic acid amplification and/or taggant sequence detection techniques may be employed to amplify and identify the nucleic acid taggant. For example, in a PCR-based identification method, the nucleic acid, e.g., DNA taggants recovered from the object are isolated and then amplified by polymerase chain reaction (PCR) and resolved by gel electrophoresis, capillary electrophoresis, or the like. Since the nucleic acid sequence of the nucleic acid taggants of the present invention are unique and specific to the tagged object, the nucleic acid taggant will be amplified during PCR only by use of primers having specific sequences complementary to a portion of the unique taggant sequence. Through this procedure, if the examined object carries the nucleic acid taggant, the PCR procedure will amplify the extracted nucleic acid to produce known and detectable amplicons of a predetermined size and a sequence. In contrast, if the sample recovered from the examined object does not include the unique nucleic acid sequence corresponding to the taggant of the authentic object, there will likely be no amplified nucleic acid product, or if the primers do amplify the recovered nucleic acid to produce one or more random amplicons, these one or more amplicons cannot have the unique taggant nucleic acid sequence from the authentic object. Furthermore, the random amplicons derived from counterfeit articles are also of random lengths and the likelihood of producing amplicons of the exact lengths specified by the taggant-specific primers is very small. Therefore, by comparing the length and quantity of PCR amplicons, the authenticity of labeled objects can be verified, non-authentic objects can be screened and rejected, and anti-counterfeit screening purposes are then achieved. The DNA may also be amplified by any known isothermal amplification technique.

The quantity of amplicons and the lengths of the amplicons can be determined after any molecular weight or physical dimension-based separation, such as for instance and without limitation, gel electrophoresis in any suitable matrix medium for example in agarose gels, polyacrylamide gels or mixed agarose-polyacrylamide gels, or the electrophoretic separation can be in a slab gel or by capillary electrophoresis. RT-PCR and/or qPCR may also be used to detect the presence of the nucleic acid taggant via interrogation of amplicon quantity and length during amplification. In addition, the nucleic acid taggant may be identified by amplification in conjunction with any suitable specific marker sequence detection methods. Moreover, digital PCR may be utilized for highly accurate detection of nucleic acid/DNA taggants. Through the use of digital PCR, the exact amount of DNA taggant obtained from an object can be ascertained. With this data, quantification of the DNA taggant in an object is possible.

Examples have been set forth below for the purpose of illustration and to describe the best mode of the invention at the present time. The scope of the invention is not to be in any way limited by the examples set forth herein.

### EXAMPLES

### Example 1

### DNA Monolayer Calculation for a 300 nm Spherical Titanium Dioxide Particle

The number of nucleic acid molecules needed to cover a mass of 300 nm diameter titanium dioxide particles was calculated. In this example, the nucleic acid was double-stranded DNA comprised of a known 400 base pair sequence.

The surface area of a single 300 nm diameter titanium dioxide particle is calculated by the formula SA=41⁻Ir², where SA equals the surface area of a sphere and r is the sphere's radius. Applied to the subject 300 nm diameter titanium dioxide particle, the following calculation can be made: 4×3.14×(300 nm/2)². This calculation reveals that each individual 300nm diameter titanium dioxide particle has a surface area of 2,826,000 A. The total surface area of any mass 300 nm diameter titanium dioxide particle can be calculated based upon the known weight of each particle.

Since DNA is a rod-like shape, the area of a DNA molecule can be calculated by multiplying its length by its width. Here, the subject DNA molecule is 400 base pairs in length. It is known that each base pair is equal to 3.4 A. Thus, the subject DNA molecule has a length of 1,360 A. It is also known that double stranded DNA is 20 A in width. Based upon these figures, the subject 400 base pair double stranded DNA molecule has a surface area of 27,200 A.

Therefore, the number of DNA molecules necessary to create a monolayer around a single 300 nm diameter titanium dioxide particle is equal to 2,826,000 A/27,200A, which is equal to 103.90 DNA molecules. With this value known, a solution containing the precise number of DNA molecules to form a monolayer around any mass of 300 nm diameter titanium dioxide submicron particles can be calculated using the methods outlined above.

### Example 2

### Attaching and Releasing a DNA monolayer to 300 nm Diameter Titanium Dioxide Particles with HCl Pretreatment

A stock suspension containing 20mg of 300nm titanium dioxide particles per mL suspended in 10mM hydrochloric acid and water solution at pH 2 was prepared. From this stock suspension, a 5004, amount was removed. The number of DNA molecules necessary to create a monolayer around the titanium dioxide submicron particles contained in the 50 μL suspension was calculated as described above.

The number of DNA molecules necessary to form a monolayer around every 300 nm titanium dioxide particle contained in the 5004, suspension was calculated and added. The combined titanium dioxide particle suspension and DNA was then vortexed for 20 seconds and then centrifuged at 10k for one minute. The resultant supernatant was removed. The remaining solid residue comprised the titanium dioxide submicron particles contained in the 500 μL suspension coated with a monolayer of DNA. The DNA coated titanium dioxide submicron particles were allowed to completely dry. Due to the pre-treatment of the 300 nm titanium dioxide particles with hydrochloric acid at a low pH, the DNA is extremely tightly bound to the titanium dioxide particles.

For DNA extraction, the DNA-coated titanium dioxide particles were re-suspended in 100 μL of 100 mM KH₂PO₄ (monopotassium phosphate) at a pH of approximately 9.5. The sample was vortexed and heated at 95° C. for three minutes. The sample was then centrifuged at 10k for one minute. The resultant supernatant was removed and used for PCR-based analyses. After the PCR run, the PCR products were analyzed via capillary electrophoreses. DNA was successfully recovered from four different samples of DNA-coated titanium dioxide particles.

### Example 3

### Attaching DNA Taggant to Food-Grade TiO₂ and Incorporating it into a Dry Powder Film Coating System

Food-grade TiO₂ powder was provided. The TiO₂ was pre-treated with a competitive binding substance, i.e., a phosphate in a weak acid. The amount of DNA taggant needed to cover the TiO₂ particles was calculated as in Example 1. The DNA was combined with the pre-treated TiO₂ as in Example 2. The resultant DNA-TiO₂ complex was mixed with untagged TiO₂ and then incorporated into a dry powder film coating system containing polymer, plasticizer, and pigment.

A series of DNA-tagged powder film coating and un-tagged powder film coating were prepared and sent to the laboratory for blind testing.

Protocol: The samples were labeled as samples #34, #36, #40, #41, #47, #49, #51, #57, #62, #63, #65, #66 and Placebo. Five different aliquots of each sample were taken and prepared for analysis at the laboratory. For each sample preparation, 50 mg of powder was weighed into a 1.5 ml Eppendorf tube and 500 μl of DNA desorption solution (monopotassium phosphate at a pH of approximately 9.5) was added to each tube. The samples were vortexed for approximately 30 seconds, incubated at room temperature for 45 minutes, heated to 95° C. for 3 minutes and then centrifuged at 17,000 g for 5 minutes. The supernatant of each preparation was then tested using the lab-scale Step One Plus™ Real-Time PCR System (qPCR).

Results: Ct (threshold cycle) values were obtained for all reactions and the average of the five sample preparations was calculated for each sample. Based on the well-known log base two relationship between Ct and input DNA concentration, one-Ct decrease in the qPCR data corresponds to a two-fold increase in input DNA. Ct values in the 35 range are near to the detection limit relative to background. Thus, placebo controls should display Ct values of approximately 35.

Thus, the data suggest that the DNA-free placebo, plus samples #36, #49, #63 and #65 do not display significant DNA in the present assay. At the other extreme, samples #34, #40, #41, #47, #51, #57, #62 and #66 (with Ct values near to 25) display a difference in Ct between 5-9 units, indicative of a 100-fold to 1000-fold higher-input DNA concentration.

Conclusion: Samples #36, #49, #63, #65 and Placebo are indistinguishable from each other and as a set, are generally indistinguishable from background. Samples #34, #40, #41, #47, #51, #57, #62 and #66 are readily distinguishable from background and appear to contain higher amounts of DNA, with samples #62 and #66 having the highest apparent DNA concentration, reflective of 10× more DNA (3-4 fold lower Ct) than samples #34, #40 and #41 and approximately 2× more DNA (1 fold lower Ct) than samples #47, #51 and #57.

DNA was detected in the appropriate samples via qPCR and no DNA was detected in the untagged samples.

### Example 4

### Attaching DNA Taggant to Food-Grade TiO₂ and Incorporating it Into a Dry Powder Film Coating System Applied to Tablet Dosage Form

The tagged powder film coating formulations made according Example 3 were used to coat tablet dosage forms. Control samples were also prepared in which un-tagged powder film coatings were used to coat tablets. The resulting samples of tablets and tagged powder film coating were sent to the laboratory for blind testing.

### A) Testing of Tagged Powder Film Coating Formulations

Protocol: Powder film coating samples were labeled as sample #68, #69 and #70. Five different aliquots of each sample were taken and prepared for analysis at the laboratory. For each sample preparation, 50 mg of powder was weighed into a 1.5 ml Eppendorf tube and 500 μl of DNA desorption solution (monopotassium phosphate at a pH of approximately 9.5) was added to each tube. The samples were vortexed for approximately 30 seconds, incubated at room temperature for 45 minutes, heated to 95° C. for 3 minutes then centrifuged at 17,000 g for 5 minutes. The supernatant of each preparation was then tested using the lab-scale StepOnePlus™ Real-Time PCR System (qPCR).

Results: Ct (threshold cycle) values were obtained for all reactions and the average of the five sample preparations was calculated for each sample. Average Ct values for each sample were obtained. Based on the well-known log base two relationship between Ct and input DNA concentration, a one-Ct decrease in the qPCR data corresponds to a two-fold increase in input DNA. Ct values in the 35 range are near to the detection limit relative to background, thus Ct values around 35 and above can be considered to contain no measurable DNA.

Conclusion: Samples #68, #69 and #70 are all readily distinguishable from background and appear to contain high amounts of DNA, with sample #69 having the highest apparent concentration, reflective of 10X more DNA (i.e. a 3-4 fold lower Ct) than sample #68 which appears to contain the lowest DNA concentration.

### B) Testing of Tablet Samples

Protocol: Tablet samples were labeled as sample #71, #72 and #73. Five different tablets were taken from each sample pack and prepared for analysis at the laboratory. Sterile cotton tipped applicators were dipped in deionized water and used to swab one side of each tablet ten times. The tip of the cotton swab was removed and placed into the PCR reaction mixture. The samples were then tested using the MyGo Pro Real-Time PCR (qPCR) Instrument.

Results: Ct (threshold cycle) values were obtained for all reactions and the average of the five sample preparations was calculated for each sample.

Conclusion: All three samples, #71-#73 appear to contain measurable amounts of DNA taggant. DNA was detected in the appropriate powder and tablets samples via qPCR and no DNA was detected in the untagged powder and tablet samples.

### Example 5

### Spray Drying DNA Taggants onto Sodium Citrate Micron or Submicron Particles

A study was performed with a commercial spray dryer to investigate whether a sodium citrate powder with an external layer or monolayer of DNA taggants could be produced. In the study, three different formulations of spray dry slurry were created. The formulations are in the table below:

| Formulation | Sodium Citrate | DNA | Water | Total Slurry |
|---|---|---|---|---|
| # | Wt (g) | wt (g) | Wt (g) | Wt (g) |
| 1 | 500 | 5 | 937.86 | 1443.86 |
| 2 | 500 | 50 | 1021 | 1573 |
| 3 | 125 | 125 | 464.29 | 717.29 |

All three formulations produced sodium citrate power, with formation number 1 having the highest percent yield. The inlet and outlet temperatures where 380+/−50 degrees Fahrenheit and 180+/−50 degrees Fahrenheit, respectively. The slurry temperature was initially 86 degrees Fahrenheit, but was reduced to 65 degrees Fahrenheit for the addition of the DNA taggants. The formation of a recoverable and detectable layer or monolayer of DNA taggants on the sodium citrate particles was confirmed via qPCR testing.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention.

Although the invention has been described with reference to the above examples and embodiments, it is not intended that such references be constructed as limitations upon the scope of this invention except as set forth in the following claims.

1. A method of attaching a nucleic acid to an object for authentication purposes comprising:
providing a plurality of micron or submicron particles;
adding an amount of nucleic acid suspended in a solvent so that only enough nucleic acid is present to form a monolayer around each submicron particle;
spray drying the plurality of micron or submicron particles and the nucleic acid suspended in a solvent to form a plurality of micron or submicron particles with a monolayer of nucleic acid covering each submicron particle; and
attaching the nucleic acid covered micron or submicron particles to an object to be authenticated using nucleic acid amplification and/or taggant sequence detection techniques for authentication.

2. The method according to claim 1, wherein the nucleic acid is DNA.

3. The method according to claim 1, wherein the micron or submicron particles are citrate, sodium citrate, titanium dioxide, maltodextrin, hydroxypropyl methylcellulose.

4. The method of claim 1, where the solvent is water.

5. The method of claim 1, wherein the object is a pharmaceutical, nutraceutical or cosmetic.

6. A method of authenticating an object comprising:
providing plurality of micron or submicron particles;
adding an amount of nucleic acid suspended in a solvent so that only enough nucleic acid is present to form a monolayer around each submicron particle;
spray drying the plurality of micron or submicron particles and the nucleic acid suspended in a solvent to form a plurality of micron or submicron particles with a monolayer of nucleic acid covering each submicron particle;
attaching the nucleic acid covered micron or submicron particles to an object to be authenticated;
taking a sample of the object to recover the nucleic acid from the micron or submicron particles;
isolating the nucleic acid;
amplifying and identifying the nucleic acid using nucleic acid amplification and/or taggant sequence detection techniques; and
verifying the authenticity of the object by the presence of the nucleic acid.

7. The method according to claim 6, wherein the nucleic acid is DNA.

8. The method according to claim 6, wherein the micron or submicron particles are citrate, sodium citrate, titanium dioxide, maltodextrin, hydroxypropyl methylcellulose.

9. The method of claim 6, where the solvent is water.

10. The method of claim 6, wherein the object is a pharmaceutical, nutraceutical or cosmetic.

11. The method of claim 6, wherein the recovery of the nucleic acid from the micron or submicron particles is accomplished by dissolving said particles.

12. A method of authenticating a pharmaceutical or nutraceutical product comprising:
providing plurality of micron or submicron particles;
adding an amount of nucleic acid suspended in a solvent so that only enough nucleic acid is present to form a monolayer around each submicron particle;
spray drying the plurality of micron or submicron particles and the nucleic acid suspended in a solvent to form a plurality of micron or submicron particles with a monolayer of nucleic acid covering each submicron particle;
adding the nucleic acid covered micron or submicron particles to a pharmaceutical or nutraceutical product;
taking a sample of the pharmaceutical or nutraceutical product to recover the nucleic acid from the micron or submicron particles disposed on or within said product;
isolating the nucleic acid;
amplifying and identifying the nucleic acid using nucleic acid amplification and/or taggant sequence detection techniques; and
verifying the authenticity of the pharmaceutical or nutraceutical product by the presence of the nucleic acid.

13. The method according to claim 12, wherein the nucleic acid is DNA.

14. The method according to claim 12, wherein the micron or submicron particles are any suitable powdered excipient for a pharmaceutical or nutraceutical product.

15. The method according to claim 12, wherein the micron or submicron particles are citrate, sodium citrate, titanium dioxide, maltodextrin, hydroxypropyl methylcellulose.

16. The method of claim 12, where the solvent is water.

17. The method of claim 12, wherein the recovery of the nucleic acid from the micron or submicron particles is accomplished by dissolving said particles.
